(19) United States
(12) Patent Application Publication
(10) Pub. No.: US 2005/0015063 A1
(43) Pub. Date: Jan. 20, 2005

(54) Quasi-T shaped catheter for chemotherapy

(76) Inventor: Yu-Chung Chang, Kaohsiung City (TW)

Correspondence Address: LAW OFFICES OF DAVID PAI, 1001 FOURTH AVENUE, SUITE 3200, SEATTLE, WA, 98154, US

(21) Appl. No.: 10/822,419

(22) Filed: Apr. 12, 2004

(30) Foreign Application Priority Data
Jul. 18, 2003 (TW) 092119889

(51) Int. Cl. A61M 25/00

(52) U.S. Cl. 604/284

(57) ABSTRACT

Disclosed is a quasi-T shaped catheter for chemotherapy for liver malignancies. Using the quasi-T shaped catheter can provide a dual chemotherapeutic effect on liver malignancies. One limb of the quasi-T shaped catheter is inserted into the common hepatic artery to provide a trans-arterial chemotherapeutic effect and the other limb is inserted into superior pancreaticoduodenal arteries, which returns to the portal vein to provide a trans-portal chemotherapeutic effect. The quasi-T shaped catheter allows fewer portals and catheters to be used so that the medical cost can be lowered; the quasi-T shaped catheter also requires fewer portals to be embedded so that the patient's suffering can be reduced.

### BACKGROUND OF THE INVENTION

1. Field of the Invention

The present invention relates to an apparatus for medical treatment, and more particularly, to an apparatus for chemotherapy for liver malignancies.

2. Description of the Related Art

The liver is an internal organ full of blood which comes from two different blood vessel systems and differs in properties. One is the arterial blood full of oxygen coming from the hepatic artery, and the other is the venous blood full of nutrients coming from the portal vein through the alimentary tract. The two kinds of blood vessels run side by side (or parallel) around the liver lobule and the bloods from them flow through the internal tissue of the liver lobule. Subsequently, the bloods flow through the sublobular vein, into the hepatic vein and finally drain into the inferior vena cava.

Conventional chemotherapy for liver malignancies has been performed via the systemic, trans-arterial, or trans-portal delivery of anti-cancer drugs. The first method, systemic chemotherapy, directly infuses the drugs from a peripheral vein or using a pre-embedded infusion portal to the branches of the external jugular vein or the subclavian vein. The second method, trans-arterial chemotherapy, infuses the drugs directly into a pre-embedded infusion portal with a straight catheter inserted from an incised orifice of the gastroduodenal or femoral artery upward into the proper hepatic artery. The third method, trans-portal chemotherapy, uses the same pre-embedded infusion portal with a catheter cannulated into a branch of the superior mesenteric vein or the umbilical vein.

Chemotherapeutic effects of the above methods have not been satisfactory in terms of patient survival. Drug sensitivity of each anti-cancer drug to the individual tumor plays the most crucial role, however, the route of drug delivery may play an important role as well. In general, the trans-arterial and the trans-portal routes have a better responsive rate than the systemic route.

Because the liver is supplied by hepatic artery and portal vein, partial concentration of the anti-cancer drugs on the malignancies can be increased through direct delivery of the anti-cancer drugs via hepatic artery and portal vein while at the same time reducing the side effect of the anti-cancer drugs throughout the body.

Until now, there has been no method that combines both the trans-arterial and the trans-portal chemotherapies.

### BRIEF SUMMARY OF THE INVENTION

In view of the above background of the invention, there has been no method of conventional chemotherapy for liver malignancies that combines both the trans-arterial and the trans-portal chemotherapies to make the chemotherapy more satisfactory in terms of patient survival.

It is a primary object of the present invention to provide a quasi-T shaped catheter, which uses a single portal with a single catheter to add up the effects of both trans-arterial and trans-portal chemotherapies so that a better chemotherapeutic effect and survival for the patients can be achieved.

It is another object of the present invention to provide a quasi-T shaped catheter for chemotherapy for liver malignancies which is capable of using fewer portals and catheters so that the medical cost is lowered.

It is yet another object of the present invention to provide a quasi-T shaped catheter for chemotherapy for liver malignancies that requires fewer embedded portals so that the patient's suffering is reduced.

In order to achieve the above objects, the present invention provides a quasi-T shaped catheter structure having three chambers for chemotherapy for liver malignancies. The structure comprises a first chamber positioned at one limb of the quasi-T shaped catheter, a second chamber positioned at another limb of the quasi-T shaped catheter opposite to the first chamber and a third chamber positioned at the other limb of the quasi-T shaped catheter. The quasi-T shaped catheter structure is formed as an integral piece.

A chemotherapy using the quasi-T shaped catheter can provide a dual chemotherapeutic effect on primary or metastatic liver malignancies. One limb of the quasi-T shaped catheter is inserted from a longitudinally incised orifice of the gastroduodenal artery upward into the proper hepatic artery, and the other limb is inserted from the same orifice downward toward the superior pancreaticoduodenal arteries. Alternatively, two limbs of the quasi-T shaped catheter are inserted together within a sheath from the femoral artery through the abdominal aorta and the celiac artery to the common hepatic artery and then the sheath is removed to enable the quasi-T shaped catheter properly placed into the proper hepatic artery and the gastroduodenal artery. The infusion of anti-cancer drugs via the proper hepatic artery provides a trans-arterial chemotherapeutic effect, whereas the infusion via the gastroduodenal artery, which will return to the liver via the superior mesenteric vein and the portal vein, provides a trans-portal chemotherapeutic effect.

### BRIEF DESCRIPTION OF THE DRAWINGS

All the objects, advantages, and novel features of the invention will become more apparent from the following detailed description when taken in conjunction with the accompanying drawings.

FIG. 1 is a schematic diagram illustrating a preferred embodiment of the quasi-T shaped catheter according to the present invention;

FIG. 2 is a schematic diagram illustrating another preferred embodiment of the quasi-T shaped catheter according to the present invention;

FIG. 3 is a schematic diagram illustrating how a preferred embodiment of the quasi-T shaped catheter according to the present invention is operated.

### DETAILED DESCRIPTION OF THE INVENTION

The present invention relates to a quasi-T shaped catheter for chemotherapy for liver malignancies. Before the usage of the quasi-T shaped catheter is described, the structural elements of the quasi-T shaped catheter is first described in the following.

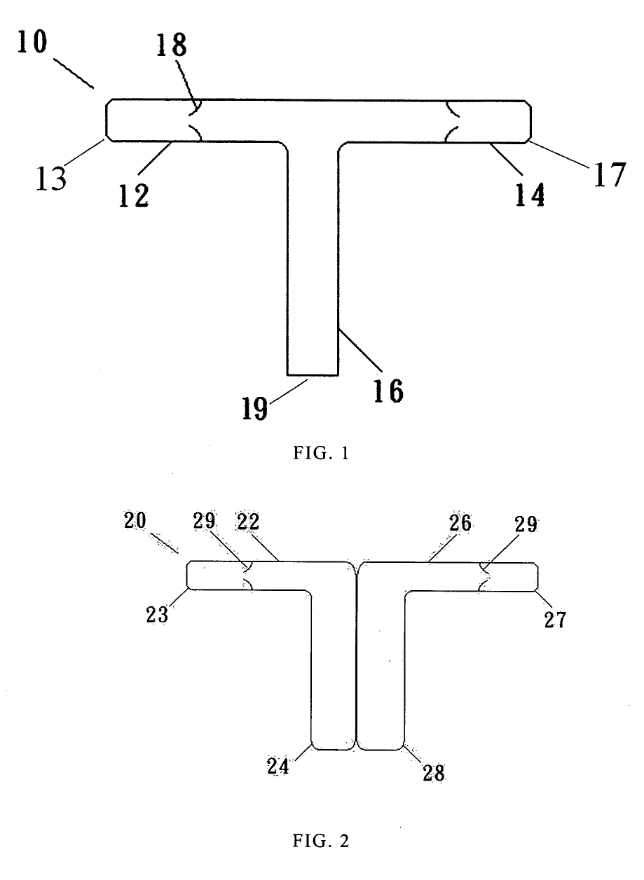

FIG. 1 is a schematic diagram illustrating a preferred embodiment of the quasi-T shaped catheter according to the present invention. The quasi-T shaped catheter 10 is formed as an integral piece and has a connecting structure. The quasi-T shaped catheter 10 comprises a first chamber 12 which is positioned at one limb of the quasi-T shaped catheter 10 and has a first portal 13 used for delivering the anti-cancer drugs, a second chamber 14 which is positioned at another limb of the quasi-T shaped catheter 10 opposite to the first chamber and has a second portal 17 used for delivering the anti-cancer drugs, and a third chamber 16 which is positioned at the other limb of the quasi-T shaped catheter 10 and has a third portal 19 used for infusing the anti-cancer drugs.

The quasi-T shaped catheter 10 can be infused with the anti-cancer drugs via the portal of the third chamber 16 and through the portals of the first chamber 12 and the second chamber 14 the anti-cancer drugs can be delivered.

The cross sections of the first chamber 12 and the second chamber 14 are preferably, though not limited to, an annular shape. The lumens of the first chamber 12 and the second chamber 14 each further comprises an anti-reflux valve 18. The length of the first chamber 12 is preferably, though not limited to, 3-10 cm. The length of the second chamber 14 is preferably, though not limited to, 3-10 cm. However, the more preferred lengths are both about 5 cm. The length of the third chamber 16 is preferably, though not limited to, 15-70 cm. It should be noticed that the outer diameter of the first chamber 12 and the second chamber 14 of the quasi-T shaped catheter 10 must be less than both the diameter of vessels of the hepatic artery and the gastroduodenal artery, and the inner diameter should be at least big enough to let the drugs flow. The diameter of the third chamber 16 is not to be so limited. The angle between the first chamber 12 and the second chamber 14 is preferably, though not limited to, about 180 degrees.

The lumen of the quasi-T shaped catheter 10 is preferably, though not necessarily, treated with an anti-coagulation coating to prevent the blood from coagulating when flowing in the lumen of the quasi-T shaped catheter 10. The quasi-T shaped catheter 10 is formed of biocompatible materials. In general, the material of the quasi-T shaped catheter 10 should be soft and elastic.

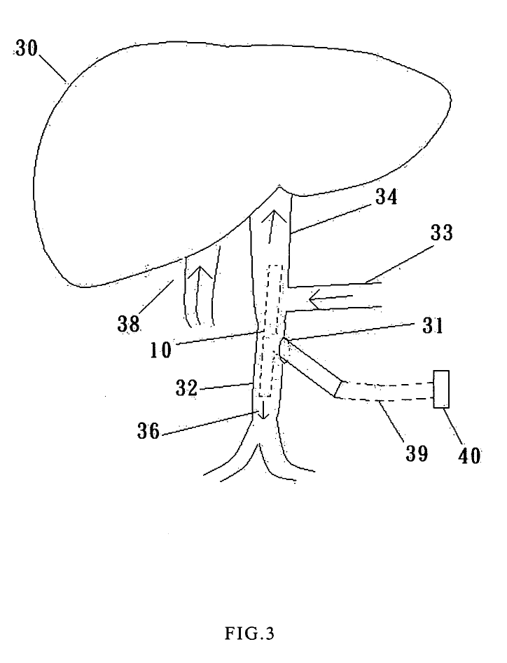

FIG. 2 is a schematic diagram illustrating another preferred embodiment of the quasi-T shaped catheter according to the present invention. The quasi-T shaped catheter 20 comprises a first catheter 22 having a first delivery portal 23 and a first infusion portal 24 formed in inverted L shape, a second catheter 26 having a second delivery portal 27 and a second infusion portal 28 formed in inverted L shape. The second infusion portal 28 of the second catheter 26 is fixed together with the first infusion portal 24 of the first catheter 22.

Therefore, the quasi-T shaped catheter 20 can be infused with the anti-cancer drugs via the first infusion portal 24 of the first catheter 22 and the second infusion portal 28 of the second catheter 26 and through the first delivery portal 23 of the first catheter 22 and the second delivery portal 27 of the second catheter 26 the anti-cancer drugs can be delivered.

The cross sections of the first catheter 22 and the second catheter 26 are preferably, though not limited to an annular shape. The lumens of the first catheter 22 and the second catheter 26 each further comprises an anti-reflux valve 29. The length of the first delivery portal 23 of the first catheter 22 is preferably, though not limited to, 3-10 cm. The length of the second delivery portal 27 of the second catheter 26 is preferably, though not limited, 3-10 cm. However, the more preferred lengths are both about 5 cm. The length of the first infusion portal 24 of the first catheter 22 is preferably, though not limited to, 15-70 cm. The length of the second infusion portal 28 of the second catheter 26 is preferably, though not limited to, 15-70 cm. It should be noticed that the outer diameter of the first catheter 22 and the second catheter 26 of the quasi-T shaped catheter 20 must be less than both the diameter of vessels of the hepatic artery and the gastroduodenal artery, and the inner diameter should be at least big enough to let the drugs flow. The angle between the first catheter 22 and the second catheter 26 is preferably, though not limited to, about 180 degrees.

The lumen of the quasi-T shaped catheter 20 is preferably, though not necessarily, treated with an anti-coagulation coating to prevent the blood from coagulating when flowing in the lumen of the quasi-T shaped catheter 20. The quasi-T shaped catheter 20 is formed of biocompatible materials. In general, the material of the quasi-T shaped catheter 20 should be soft and elastic. The back ends of the first catheter 22 and the second catheter 26 are connected to a common infusion portal.

FIG. 3 is a schematic diagram illustrating how a preferred embodiment of the quasi-T shaped catheter according to the present invention is operated. A chemotherapy using the quasi-T shaped catheter of the invention can provide a dual chemotherapeutic effect on primary or metastatic liver malignancies. The following is a description of the embodiment of the quasi-T shaped catheter 10 illustrated in FIG. 1. Notice that the organs and the vessels in FIG. 3 are not in actual scale, and the arrow indicates the direction of the blood flow. The first chamber 12 of the quasi-T shaped catheter 10 is inserted from a longitudinally incised orifice 31 of the gastroduodenal artery 32 upward into the proper hepatic artery 34, and the second chamber 14 of the quasi-T shaped catheter 10 is inserted from the same orifice 31 downward toward the superior pancreaticoduodenal artery 36. The superior pancreaticoduodenal artery 36 is divided into anterior and posterior pancreaticoduodenal arteries. Another embodiment is: both the first chamber 12 and the second chamber 14 of the quasi-T shaped catheter 10 are confined in a sheath while being inserted from a vessel, such as the femoral artery, through the abdominal aorta (not shown) and the celiac artery (not shown) to the common hepatic artery 33 and then the sheath is withdrawn and the two limbs of the quasi-T shaped catheter are placed into the proper hepatic artery 34 and the gastroduodenal artery 32. The infusion via the proper hepatic artery can provide a trans-arterial chemotherapeutic effect, whereas the infusion via the gastroduodenal artery, which will return to the liver via the superior mesenteric vein and the portal vein, provides a trans-portal chemotherapeutic effect. The third chamber 16 of the quasi-T shaped catheter 10 is connected to an infusion portal 40 through a pipe 39 to provide a route for infusing the drugs.

The usage of the quasi-T shaped catheter 20 in FIG. 2 is similar to the above description. However, it should be noticed that the first infusion portal 24 of the first catheter 22 and the second infusion portal 28 of the second catheter 26 are connected to a common infusion portal 40.

To sum up, it is a primary advantage of the present invention to provide a quasi-T shaped catheter, which uses a single portal with a single catheter to add up the effects of both trans-arterial and trans-portal chemotherapies.

It is another advantage of the present invention that using the quasi-T shaped catheter of the invention can provide a better chemotherapeutic effect and survival for the patients.

It is another advantage of the present invention that using the quasi-T shaped catheter of the invention needs fewer portals and catheters so that the medical cost is lowered.

It is still another advantage of the present invention that using a quasi-T shaped catheter of the invention requires fewer embedded portals so that the patient's suffering is reduced.

It is still another advantage of the present invention that a quasi-T shaped catheter of the invention is not easy to dislodge, which occurred in 26.4% of patients using conventional straight tip.

Until now, no such quasi-T shaped catheters have ever been considered for chemotherapeutic purposes. Therefore, neither before did such quasi-T shaped catheters exist nor have they been applied. It is an innovative idea to combine both the trans-arterial chemotherapy and the trans-portal chemotherapy for liver malignancies and to develop the quasi-T shaped catheter for this purpose.

Although the invention has been explained in relation to its preferred embodiments, it is to be understood that many other modifications and variations can be made without departing from the spirit and scope of the invention as hereinafter claimed.

1. A quasi-T shaped catheter for delivering anti-cancer drugs to the liver for chemotherapy, comprising:
a first chamber positioned at one limb of said quasi-T shaped catheter and having a first portal for delivering the anti-cancer drugs;
a second chamber positioned at another limb of said quasi-T shaped catheter opposite to the first chamber and having a second portal for delivering the anti-cancer drugs; and
a third chamber positioned at the other limb of the quasi-T shaped catheter and having a third portal for infusing the anti-cancer drugs;
wherein said quasi-T shaped catheter is formed as an integral piece and said first, second and third chambers are in communication with each other, and the first portal and the second portal are to be inserted into the vessels leading respectively to the hepatic artery and the portal vein.

2. The quasi-T shaped catheter of claim 1, wherein the cross sections of said first chamber and said second chamber are of an annular shape.

3. The quasi-T shaped catheter of claim 1, wherein said first chamber and said second chamber further comprises an anti-reflux valve.

4. The quasi-T shaped catheter of claim 1, wherein said first chamber has a length between 3 and 10 cm.

5. The quasi-T shaped catheter of claim 1, wherein said second chamber has a length between 3 and 10 cm.

6. The quasi-T shaped catheter of claim 1, wherein an anti-coagulation coating is applied to its interior surface.

7. The quasi-T shaped catheter of claim 1, wherein said quasi-T shaped catheter is formed of biocompatible materials.

8. A quasi-T shaped catheter for delivering anti-cancer drugs to the hepatic artery and portal vein system of the liver for chemotherapy, comprising:
a first catheter having a first delivery portal and a first infusion portal formed in an inverted L shape; and
a second catheter having a second delivery portal and a second infusion portal formed in an inverted L shape;
wherein the second infusion portal of the second catheter is fixed together with the first infusion portal of the first catheter, and the first infusion portal and the second infusion portal are to be inserted into the vessels leading respectively to the hepatic artery and the portal vein.

9. The quasi-T shaped catheter of claim 8, wherein the cross sections of said first catheter and said second catheter are of an annular shape.

10. The quasi-T shaped catheter of claim 8, wherein said first catheter and said second catheter each comprises an anti-reflux valve.

11. The quasi-T shaped catheter of claim 8, wherein said first catheter has a length between 3 and 10 cm.

12. The quasi-T shaped catheter of claim 8, wherein said second catheter has a length between 3 and 10 cm.

13. The quasi-T shaped catheter of claim 8, wherein an anti-coagulation coating is applied to its interior surface.

14. The quasi-T shaped catheter of claim 8, wherein said quasi-T shaped catheter is formed of biocompatible materials.

15. The quasi-T shaped catheter of claim 8, wherein said first catheter and said second catheter are connected to a common infusion portal.
